(19) United States
(12) Patent Application Publication
(10) Pub. No.: US 2006/0108910 A1
(43) Pub. Date: May 25, 2006

(54) Device for generating radiation

(76) Inventors: Thomas Justel, Aachen (DE); Walter Mayr, Alsdorf (DE); Peter J. Schmidt, Aachen (DE); Helmut Blankefort, Lathen (DE)

Correspondence Address: Corporate Patent Counsel;Philips Electronics North America Corporation, P O Box 3001, Briarcliff Manor, NY, 10510, US

(21) Appl. No.: 10/524,658

(22) PCT Filed: Aug. 14, 2003
(86) PCT No.: PCT/IB03/03639

§ 371 Date: Sep. 15, 2005

(30) Foreign Application Priority Data
Aug. 22, 2002 (DE) 102 38 399.5

(51) Int. Cl. C09K 11/66; H01J 1/62

(52) U.S. Cl. 313/486; 252/301.4 F

(57) ABSTRACT

The invention relates to a device for generating radiation by means of excimer discharge, equipped with an at least partly UV-transparent discharge vessel (1), the discharge chamber (2) of which is filled with a gas filling, with means for igniting and maintaining an excimer discharge (4, 5) in the discharge chamber and with a coating (3) comprising a light-emitting compound. The light-emitting compound has the following composition: (Ca_{1-x-2y}Srₓ)Li₂Si_{1-z}Ge_{z}O₄:Ln_{y}M_{y}, wherein Ln is a cation selected from the group Ce³⁺, Pr³⁺, Sm³⁺, Eu³⁺, Gd³⁺, Tb³⁺, Dy³⁺, Er³⁺, Tm³⁺ and Yb³⁺, and M is a cation selected from the group Na⁺, K⁺ and Rb⁺, 0 £x £0.1, 0.001 £y £0.2 and 0 £z £1. The coating (3) preferably comprises Ca_{1-2y}Li₂SiO₄:Pr_{y}Na_{y} with 0.001 £y £0.2. A device with a coating (3) comprising Ca_{1-2y}Li₂SiO₄:Pr_{y}Na_{y} with 0.001 £y £0.2 may be used for disinfection purposes.

The invention relates to a device for generating radiation by means of excimer discharge, equipped with an at least partly UV-transparent discharge vessel, the discharge space of which is filled with a gas filling, with means for igniting and maintaining an excimer discharge in the discharge spacer and with a coating comprising a light-emitting compound. The invention further relates to the use of the device and to a light-emitting compound.

Phosphors are materials capable of luminescence, which emit light when excited. Exciting energy forms that are possibilities here are electromagnetic radiation such as y rays, X-rays, UV light or visible light, corpuscular rays such as α rays or β rays, electrical fields (electroluminescence) or mechanical effects (triboluminescence).

The use of phosphors is extremely varied and depends on the type of excitation and the wavelength of the emitted light. Thus, phosphors that luminesce on excitation with electron beams are used in, for example, television and computer screens, oscillograph tubes or image converters.

Phosphors that luminesce on excitation with X-rays are used to manufacture radioscopy screens and X-ray image amplifiers for medical purposes. The phosphors used must have base materials that are capable of strongly absorbing X-rays.

Phosphors that luminesce on excitation with UV radiation are used in, for example, gas discharge lamps or plasma screens.

Although suitable phosphors are available for virtually all application areas, there are still some in which the phosphors used are not yet optimal.

Furthermore, it is necessary to make phosphors available for new or special applications and for new technologies. One example of a special application is the use of xenon gas discharge lamps for disinfection purposes. Using a xenon gas discharge lamp emitting UV-C light, pathogenic microorganisms present in water, such as bacteria, fungi, viruses or protozoans, can be rendered harmless. A xenon gas discharge lamp for water disinfection is known from EP 1 048 620.

In accordance with DIN 5031-10, the maximum photobiologically effective UV radiation lies at around 265 nm.

It is therefore an object of the present invention to provide a device for generating radiation with an emission spectrum that is simple and can be optimally adjusted to suit the desired application.

It is, in particular, an object of the present invention to provide a device for generating radiation with an emission spectrum having a large overlap with DIN 5031-10.

This object is achieved by means of a device for generating radiation by means of excimer discharge, equipped with an at least partly UV-transparent discharge vessel, the discharge chamber of which is filled with a gas filling, with means for igniting and maintaining an excimer discharge in the discharge chamber and with a coating comprising a light-emitting compound of the following composition:
(Ca_{1-x-2y}Srₓ)Li₂Si_{1-z}Ge_{z}O₄:Ln_{y}M_{y},

wherein Ln is a cation selected from the group Ce³⁺, Pr³⁺, Sm³⁺, Eu³⁺, Gd³⁺, Tb³⁺, Dy³⁺, Er³⁺, Tm³⁺ and Yb³⁺,

and M is a cation selected from the group Na⁺, K⁺ and Rb⁺,

0≦x≦0.1, 0.001≦y≦0.2 and 0≦z≦1.

The light-emitting compounds activated with Ce³⁺, Pr³⁺, Sm³⁺, Eu³⁺, Gd³⁺, Tb³⁺, Dy³⁺, Er³⁺, Tm³⁺ or Yb³⁺ can all be efficiently excited using (V)UV light and have high absorption efficiencies, particularly at wavelengths in the range of xenon excimer radiation.

The device for generating radiation as claimed in claim 2 has an emission spectrum with emission maxima located at 255 and 265 nm, which is in the photobiologically effective range.

The invention also relates to the use of a device in accordance with the invention for disinfection purposes, in particular for the disinfection of water, air or surfaces.

The invention further relates to a light-emitting compound of the following composition:
(Ca_{1-x-2y}Srₓ)Li₂Si_{1-z}Ge_{z}O₄:Ln_{y}M_{y},
wherein Ln is a cation selected from the group Ce³⁺, Pr³⁺, Sm³⁺, Eu³⁺, Gd³⁺, Tb³⁺, Dy³⁺, Er³⁺, Tm³⁺ and Yb³⁺,

and M is a cation selected from the group Na⁺, K⁺ and Rb⁺,

0≦x≦0.1, 0.00≦y≦0.2 and 0≦z≦1.

The invention will be further described with reference to examples of embodiments shown in the drawings, to which, however, the invention is not restricted.

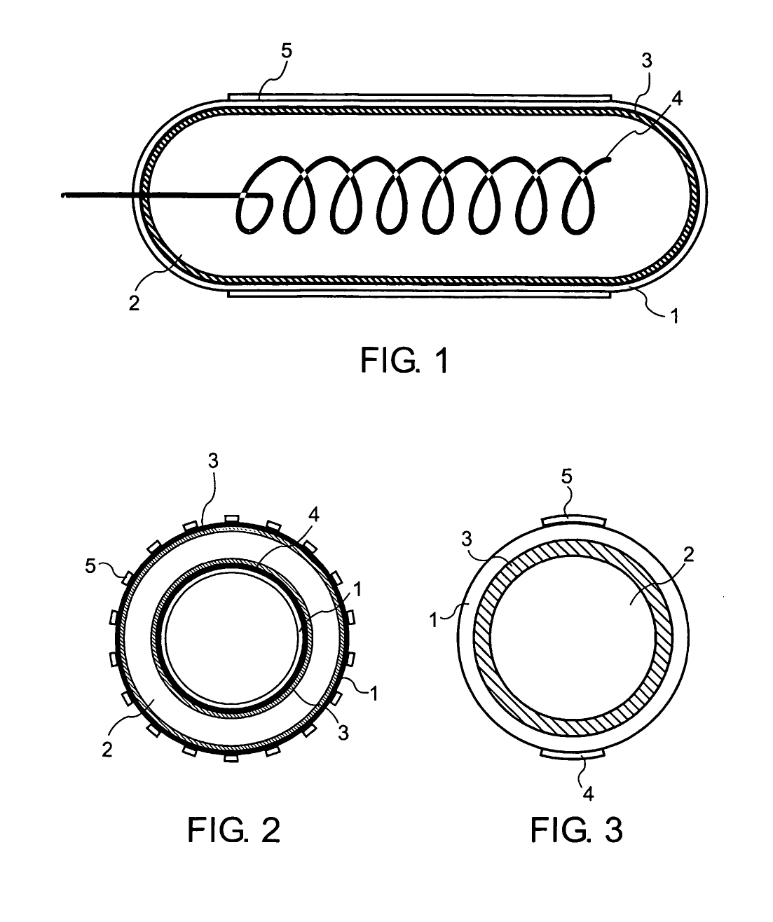

FIG. 1 shows, in longitudinal cross-section, a device for generating radiation.

FIG. 2 shows, in cross-section, a further device for generating radiation.

FIG. 3 shows, in cross-section, a yet further device for generating radiation.

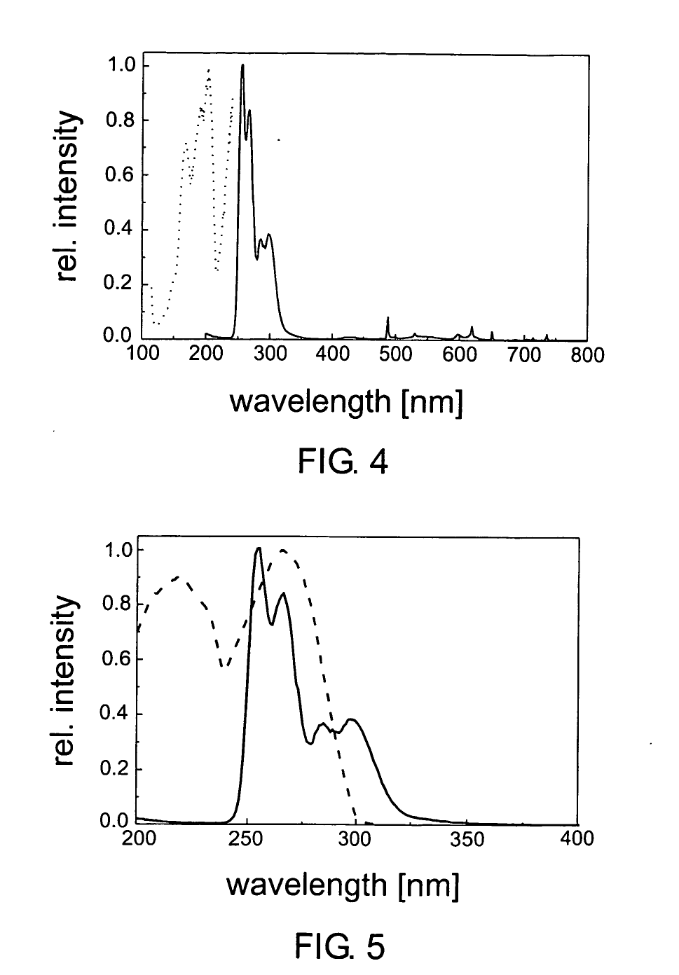

FIG. 4 shows the excitation and emission spectrum of Ca_{1-2y}Li₂SiO₄:Pr_{y}Na_{y} with 0.001≦y≦0.2.

FIG. 5 shows DIN 5031-10 and the emission spectrum of Ca_{1-2y}Li₂SiO₄:Pr_{y}Na_{y} with 0.001≦y≦0.2.

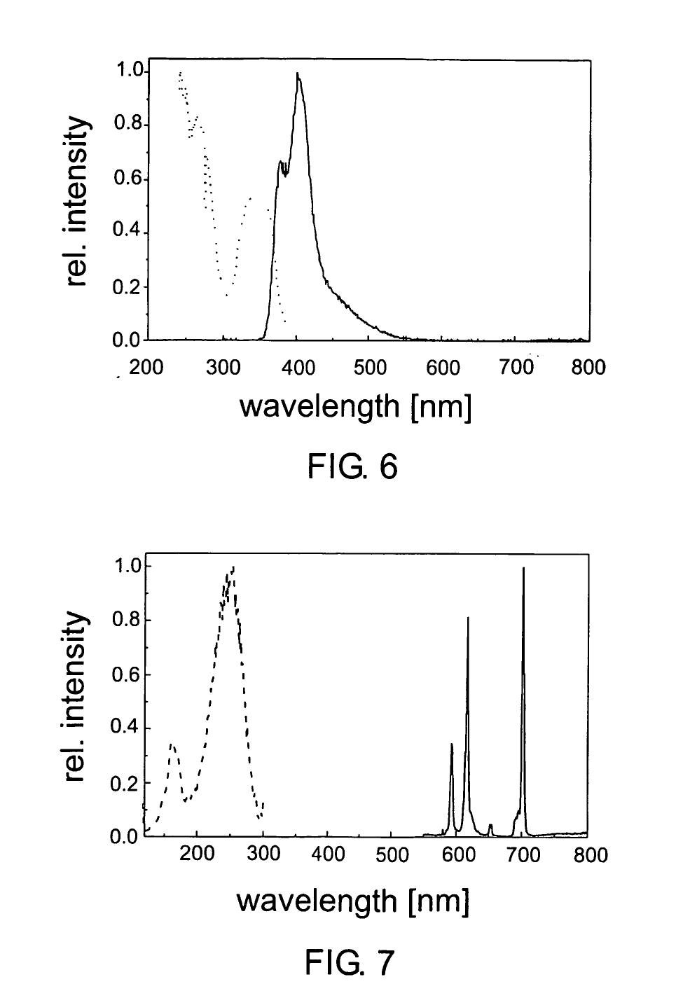

FIG. 6 shows the excitation and emission spectrum of Ca_{1-2y}Li₂SiO₄:Ce_{y}Na_{y} with 0.001≦y≦0.2.

FIG. 7 shows the excitation and emission spectrum of Ca_{1-2y}Li₂SiO₄:Eu_{y}Na_{y} with 0.001≦y≦0.2.

A device in accordance with the invention for generating radiation by means of excimer discharge is equipped with an at least partly UV-transparent discharge vessel 1, the discharge space 2 of which is filled with a gas filling, and with a coating 3 comprising a light-emitting compound. In addition, the device is equipped with means for igniting and maintaining the excimer discharge.

A multiplicity of designs, such as panels, simple tubes, coaxial tubes, discharge tubes that are straight, U-shaped, circular, spiraled or cylindrical or of some other shape, may be used for the discharge vessel 1. The design hereby depends largely on the planned application of the device.

A typical design for a device for generating radiation is the shape as shown in FIG. 1. As the means for igniting and maintaining the excimer discharge, this design comprises inter alia electrodes of first and second kinds 4, 5. Inserted concentrically into the discharge vessel 1 is a spiraled wire. This forms the first, inner electrode 4 of the device. The outer glass is covered with narrow-gauge wire netting, forming the second, outer electrode 5. Discharge vessel 1 is sealed to be gas-tight. The discharge space 2 contains a gas filling and on the inner walls of discharge vessel 1 is a coating 3, which comprises a light-emitting compound. The two electrodes 4, 5 are connected to the two poles of an alternating current source. The electrode geometry together with the pressure in discharge vessel 1 and the gas composition are matched to the data for the alternating current source.

A different design, which is particularly suitable for disinfection purposes, is the coaxial shape in FIG. 2, carrying a medium to be disinfected, such as water or air, centrally. The discharge vessel 1 comprises two coaxial glass components with a gas-tight connection to a hollow sleeve. The annular clearance between the two coaxial glass components forms the discharge chamber 2 and is provided with a gas filling. The fluid medium for disinfection can flow through the inner tube, on the inner wall of which a transparent electrode of the first kind 4 is arranged. The medium to be disinfected may also be located outside the outer tube. The outer glass is covered with narrow-gauge wire netting, forming the outer, second electrode 5. The power supply is from an alternating current source connected to these two electrodes. The inner surfaces of the hollow sleeve have a coating 3 with a light-emitting compound.

In a further possible design as shown in FIG. 3, the device comprises a cylindrical discharge vessel 1 made of glass, with a gas filling, on the walls of which the electrode of the first kind 4 and the electrode of the second kind 5 are arranged externally, electrically insulated from each other. The strip-shaped electrodes 4, 5 extend over the entire length of discharge vessel 1, wherein their long sides face each other, leaving two gaps free. The coating 3 with the light-emitting substance is applied to the inner walls of discharge vessel 1.

The means for igniting and maintaining an excimer discharge comprise electrodes of first and second kinds 4, 5. Electrodes 4, 5 comprise a metal, for example aluminum or silver, a metal alloy or a transparent, conductive, inorganic compound, e.g. ITO. These may take the form of a coating, a bonded-on film, bonded-on strips of film, wire or wire netting.

Discharge vessel 1 is equipped with a gas filling of xenon, krypton, argon, neon, helium or a gas mixture with at least one of these gases. The gas filling will preferably contain oxygen-free xenon or a gas mixture containing xenon.

Coating 3 comprises a light-emitting compound with the following composition:
(Ca_{1-x-2y}Srₓ)Li₂Si_{1-z}Ge_{z}O₄:Ln_{y}M_{y},

wherein Ln is a cation selected from the group Ce³⁺, Pr³⁺, Sm³⁺, Eu³⁺, Gd³⁺, Tb³⁺, Dy³⁺, Er³⁺, Tm³⁺ and Yb³⁺,

and M is a cation selected from the group Na⁺, K⁺ and Rb⁺,

0≦x≦0.1, 0.00≦y≦0.2 and 0≦z≦1.

The host lattice (Ca_{1-x-2y}Srₓ)Li₂Si_{1-z}Ge_{z}O₄ of the light-emitting compound crystallizes in a tetragonal crystal system. Through doping with trivalent metal cations, phosphors having different emission properties depending on the metal cation used are obtained. The trivalent metal cations are incorporated into the divalent calcium positions. For charging compensation, monovalent cations are also incorporated into the crystal lattice. Also advantageous in the case of this host lattice is the fact that the dopants can be incorporated into the host lattice at high concentrations.

The light-emitting compound preferably has a composition of:
Ca_{1-2y}Li₂SiO₄:Pr_{y}Na_{y}, Ca_{1-2y}Li₂SiO₄:Ce_{y}Na_{y}, Ca_{1-2y}Li₂SiO₄:Eu_{y}Na_{y},
Ca_{1-2y}Li₂SiO₄:Tb_{y}Na_{y} or Ca_{1-2y}Li₂SiO₄:Nd_{y}Na_{y} with 0.001≦y≦0.2 in each case. The light-emitting substance is selected depending on the application. If the device is to be used for disinfection purposes, e.g. for disinfection of water, air or surfaces, Ca_{1-2y}Li₂SiO₄:Pr_{y}Na_{y} with 0.001≦y≦0.2 is preferably used as the light-emitting substance.

Alternatively, coating 3 may also comprise an organic or inorganic binder or binder composition. Furthermore, coating 3 or the light-emitting compound itself may also be protected against the discharge attack with a protective layer.

If an alternating voltage is applied to the electrodes 4, 5, a silent electrical charge is ignited in the preferred, xenon-containing filler gas. As a result, xenon excimers, i.e. molecules that are stable only in the excited state, are formed in the plasma.
Xe+Xe*=Xe₂*

The excitation energy is released again as UV radiation with a wavelength of λ=140 to 190 nm. This conversion of electron energy into UV radiation takes place extremely efficiently. The UV photons generated are absorbed by the light-emitting compound, and some of the excitation energy is released again in an area of the spectrum with a longer wavelength. The absorption coefficient of the light-emitting compounds activated with Ce³⁺, Pr³⁺, Sm³⁺, Eu³⁺, Gd³⁺, Tb³⁺, Dy³⁺, Er³⁺, Tm³⁺ or Yb³⁺ is especially great for the wavelengths in the area of xenon radiation, and the quantum yield is high. The host lattice influences the precise position of the energy level of the activator cation and, as a result, influences the emission spectrum.

In order to produce the light-emitting compound, appropriate quantities of the starting compounds for the host lattice, Li₂SiO₃, Li₂GeO₃, CaCO₃ and SrCO₃, are mixed in water with the appropriate quantities of a salt of the trivalent metal cation Ln³⁺ and a salt of the monovalent charge compensator M+. The water is subsequently removed. The reaction mixture obtained is dried and annealed multiple times at temperatures between 700 and 900° C.

Preferably used as salts of the trivalent metal cations are the nitrates Ln(NO₃)₃ with Ln selected from the group Ce³⁺, Pr³⁺, Sm³⁺, Eu³⁺, Gd³⁺, Tb³⁺, Dy³⁺, Er³⁺, Tm³⁺ and Yb³⁺. Preferably used as salts of the charge compensator are the carbonates M₂CO₃ with M selected from the group Na⁺, K⁺ and Rb⁺.

The light-emitting compound is applied to the vessel walls of the discharge vessel 1 using a flow coating method. The coating suspensions for the flow coating method contain water or an organic compound such as butyl acetate as the solvent. The suspension is stabilized and influenced in its rheological properties by the addition of auxiliary agents such as stabilizers, liquefiers or cellulose derivatives. The suspension of the light-emitting compound is applied as a thin layer to the vessel walls of discharge vessel 1, is dried and baked at 600° C. Discharge vessel 1 is then evacuated in order to remove all gaseous impurities, especially oxygen. The vessel is then filled with xenon with a gas pressure of approximately 200 to 300 mbar, and sealed.

The uses of the device described are varied, and depend primarily on the emission spectrum of the device.

The device described, with Ca_{1-2y}Li₂SiO₄:Pr_{y}Na_{y} with 0.001≦y≦0.2 as the light-emitting compound, emits light in the UV light range, and is very suitable for high yield photolytic reactors. FIG. 4 shows the excitation and emission spectrum of the light-emitting compound. Since the emitted radiation has a narrow-band spectrum, the device in accordance with the invention may be advantageously used to undertake wavelength-selective photoreactions.

In addition, technical photochemical processes (e.g. photochlorination, photobromination, photosulfochlorination) may be undertaken more efficiently with the device in accordance with the invention.

A preferred use of this device in accordance with the invention relates to water and wastewater treatment, in which polluted water is the fluid to be treated. Examples of treatment that can be mentioned are: a) disinfection, b) degradation of pollutants, pigments or odors.

The device in accordance with the invention, with Ca_{1-2y}Li₂SiO₄:Pr_{y}Na_{y} with 0.001≦y≦0.2 as the light-emitting compound, may also be used for the sterilization of other fluids and solvents.

Phosphor Ca_{1-2y}Li₂SiO₄:Ce_{y}Na_{y} with 0.001≦y≦0.2 may also be advantageously used in a device for X-ray computer tomography.

A device for X-ray computer tomography is equipped with at least one X-ray tube and at least one X-ray detector. Usually, the device for X-ray computer tomography comprises an X-ray detector setup. An X-ray detector comprises a scintillator and a photodiode. The scintillator and photodiode are optically connected. X-rays falling onto the scintillator in the form of radiation are converted into light. The photodiode is then irradiated by the light emitted by the scintillator. As a result, information concerning the intensity of X-ray radiation can be obtained as electrical information from a photodiode.

In a device in accordance with the invention for X-ray computer tomography, the scintillator comprises Ca_{1-2y}Li₂SiO₄:Ce_{y}Na_{y} with 0.001≦y≦0.2. The scintillator is generally produced by the compression method. A scintillator of this kind is secured on the photodiode using, for example, a transparent adhesive. On excitation with X-ray radiation, Ca_{1-2y}Li₂SiO₄:Ce_{y}Na_{y} with 0.001≦y≦0.2 exhibits an emission spectrum with a maximum at 400 nm. FIG. 6 shows the excitation and emission spectrum of the light-emitting compound. The phosphor is especially suitable for proving or for converting X-ray radiation since, here too, the maxima of the photomultipliers used in devices for X-ray computer tomography are based on Cs3Sb, bi-alkali (S22) or multi-alkali cathodes. Furthermore, with a decay time τ_{1/e} of 36 ns, the phosphor has a shorter decay time (τ_{1/e}=40 ns) than the frequently used scintillator Lu₂SiO₅:Ce.

The light-emitting compound Ca_{1-2y}Li₂SiO₄:Eu_{y}Na_{y} with 0.001≦y≦0.2 may be advantageously used as, for example, red-emitting phosphor in a plasma screen. FIG. 7 shows the excitation and emission spectrum of the light-emitting compound.

### EMBODIMENT EXAMPLE 1

In order to produce Ca_{0.98}Li₂SiO₄:Pr_{0.01},Na_{0.01}, we mixed 25.0 g (278 mmol) Li₂SiO_{3,147} mg (1.39 mmol) Na₂CO₃, 27.3 g (272 mmol) CaCO₃ and 1.21 g (2.78 mmol) Pr(NO₃)₃.6H₂O in demineralized H₂O and suspended it. The water was removed by distillation and the residue obtained was dried. The reaction product was then annealed once in air at 700° C. for 2 h, and twice in a CO atmosphere at 850° C. for 12 h each time. The light-emitting compound obtained was washed multiple times with water and ethanol, dried and milled on a roller mill for several hours. The powder obtained had an average particle size of 3 μm. The absorption and emission spectrum of this light-emitting compound is shown in FIG. 4. FIG. 5 shows the DIN 5031-10 (broken line) in comparison with the emission spectrum of Ca_{0.98}Li₂SiO₄:Pr_{0.01},Na_{0.01} (solid line).

### EMBODIMENT EXAMPLE 2

In order to produce Ca_{0.96}Li₂SiO₄:Ce_{0.02},Na_{0.02}, we mixed 9.00 g (100 mmol) Li₂SiO₃, 106 mg (1.00 mmol) Na₂CO₃, 9.61 g (96.0 mmol) CaCO₃ and 869 mg (2.00 mmol) Pr(NO₃)₃.6H₂O in demineralized H₂O and suspended it. The water was removed by distillation and the residue obtained was dried. The reaction product was then annealed once in air at 700° C. for 2 h, and twice in a CO atmosphere at 850° C. for 12 h each time. The light-emitting compound obtained was washed multiple times with water and ethanol, dried and milled on a roller mill for several hours. The light-emitting compound obtained had a color point x, y of x=0.160 and y=0.069. The lumen equivalent of the light-emitting compound was 30 lm/W. The absorption coefficient at 254 nm of the light-emitting compound was 93% and the quantum efficiency at this wavelength was 80%. The absorption and emission spectrum for this light-emitting compound is shown in FIG. 6.

### EMBODIMENT EXAMPLE 3

In order to produce Ca_{0.99}Li₂SiO₄:Eu_{0.01},Na_{0.01}, we mixed 20.1 g (218 mmol) Li₂SiO₃, 90.0 mg (2.18 mmol) NaF, 12.0 g (214 mmol) CaO and 933 mg (2.18 mmol) Eu(NO₃)₃.5H₂O in demineralized H₂O and suspended it. The water was removed by distillation and the residue obtained was dried. The reaction product was then annealed once in air at 700° C. for 2 h, and once at 850° C. for 12 h. The light-emitting compound obtained was ground in a mortar and again annealed at 850° C. for 6 h. The absorption and emission spectrum for this light-emitting compound is shown in FIG. 7.

### EMBODIMENT EXAMPLE 4

In order to produce Ca_{0.98}Li₂SiO₄:Tb_{0.01},Na_{0.01}, we mixed 20.1 g (218 mmol) Li₂SiO₃, 90.0 mg (2.18 mmol) NaF, 12.0 g (214 mmol) CaO and 987 mg (2.18 mmol) Tb(NO₃)₃.6H₂O in demineralized H₂O and suspended it. The water was removed by distillation and the residue obtained was dried. The reaction product was then annealed once in air at 700° C. for 2 h, and once at 850° C. for 12 h. The light-emitting compound obtained was ground in a mortar and again annealed at 850° C. for 6 h.

### EMBODIMENT EXAMPLE 5

In order to produce Ca_{0.98}Li₂SiO₄:Nd_{0.01},Na_{0.01}, we mixed 9.82 g (109 mmol) Li₂SiO₃, 46.0 mg (1.09 mmol) NaF, 6.00 g (107 mmol) CaO and 480 mg (1.09 mmol) Nd(NO₃)₃.6H₂O in demineralized H₂O and suspended it. The water was removed by distillation and the residue obtained was dried. The reaction product was then annealed once in air at 700° C. for 2 h, and once at 850° C. for 12 h. The light-emitting compound obtained was ground in a mortar and again annealed at 850° C. for 6 h.

### EMBODIMENT EXAMPLE 6

Firstly, a suspension of Ca_{0.98}Li₂SiO₄:Pr_{0.01},Na_{0.01} was produced in butyl acetate with nitrocellulose as the binder. The suspension was applied to the internal walls of a tube made of synthetic quartz (Suprasil™) with an inside diameter of 5 mm using a flow coating method. The depth of coating 3 corresponded to a mass-per-unit-area for the light-emitting compound of 3 mg/cm². The binder was burnt out at a temperature below 580° C. The device was filled with xenon at a gas pressure of 200 to 300 mbar and then sealed. Two electrodes 4, 5 made of aluminum foil, were bonded diagonally onto the outside walls of the device.

The device was operated with alternating current with a square-wave characteristic at an amplitude of 6 kV and a frequency of 25 kHz.

### EMBODIMENT EXAMPLE 7

The discharge vessel 1 of the device in accordance with embodiment example 2 comprised a cylindrical tube made of synthetic quartz (Suprasil™) with a thickness of 0.7 mm and a diameter of 50 mm. It was filled with xenon at a pressure of 200 mbar. A spirally wound inner electrode 4 made of metal wire was arranged in the axis of the tube. Six strips of silver film were bonded onto the outside walls of the discharge vessel, in parallel with the inner electrode, as the outer electrodes 5. The inside surface of the outside walls was coated, wherein coating 3 comprised Ca_{0.98}Li₂SiO₄:Pr_{0.01},Na_{0.01} as the light-emitting compound.

The device was operated with alternating current with a square-wave characteristic at an amplitude of 6 kV and a frequency of 25 kHz.

### EMBODIMENT EXAMPLE 8

The discharge vessel 1 of the device in accordance with embodiment example 8 comprised two coaxial tubes made of synthetic quartz (Suprasil™) with a thickness of 0.7 mm, an inside diameter of 50 mm and an outside diameter of 40 mm respectively. The two tubes were joined together at both ends in such a way that they formed a gas-tight hollow sleeve. Discharge vessel 1 was filled with xenon at a pressure of 200 mbar. The wall of the hollow sleeve facing the tube axis was covered, on its side facing the tube axis, with a layer of aluminum, forming the first electrode 4, and onto the wall of the hollow sleeve facing away from the tube axis, on its side facing away from the tube axis, were bonded six strips of silver film, parallel with the tube axis, forming the second electrode 5. The internal surfaces of the hollow sleeve were coated, wherein coating 3 comprised Ca_{0.98}Li₂SiO₄:Pr_{0.01},Na_{0.01} as the light-emitting compound.

The device was operated with alternating current with a square-wave characteristic at an amplitude of 6 kV and a frequency of 25 kHz.

1. A device for generating radiation by means of excimer discharge, equipped with an at least partly UV-transparent discharge vessel (1), the discharge space (2) of which is filled with a gas filling, with means for igniting and maintaining an excimer discharge (4, 5) in the discharge space, and with a coating (3) comprising a light-emitting compound of the following composition:
(Ca_{1-x-2y}Srₓ)Li₂Si_{1-z}Ge_{z}O₄:Ln_{y}M_{y},
wherein Ln is a cation selected from the group Ce³⁺, Pr³⁺, Sm³⁺, Eu³⁺, Gd³⁺, Tb³⁺, Dy³⁺, Er³⁺, Tm³⁺ and Yb³⁺,
and M is a cation selected from the group Na⁺, K⁺ and Rb⁺, with
0≦x≦0.1, 0.001≦y≦0.2 and 0≦z≦1.

2. A device as claimed in claim 1, characterized in that the coating (3) is equipped with a light-emitting compound of the following composition:
Ca_{1-2y}Li₂SiO₄:Pr_{y}Na_{y} with 0.001≦y≦0.2.

3. A use of a device as claimed in claim 1 or 2 for disinfection purposes.

4. A use of a device as claimed in claim 1 or 2 for disinfecting water, air, or surfaces.

5. A light-emitting compound of the following composition:
(Ca_{1-x-2y}Srₓ)Li₂Si_{1-z}Ge_{z}O₄:Ln_{y}M_{y},
wherein Ln is a cation selected from the group Ce³⁺, Pr³⁺, Sm³⁺, Eu³⁺, Gd³⁺, Tb³⁺, Dy³⁺, Er³⁺, Tm³⁺ and Yb³⁺,
and M is a cation selected from the group Na⁺, K⁺ and Rb⁺, with
0≦x≦0.1, 0.001≦y≦0.2 and 0≦z≦1.

6. A light-emitting compound of the following composition:
Ca_{1-2y}Li₂SiO₄:Pr_{y}Na_{y} with 0.001≦y≦0.2.
